(19) United States
(12) Patent Application Publication
(10) Pub. No.: US 2019/0030519 A1
(43) Pub. Date: Jan. 31, 2019

(54) Process for preparing a hierarchical zeolite catalyst for aromatization of C5-C9 alkane

(71) Applicant: PTT Global Chemical Public Company Limited, Bangkok (TH)

(72) Inventors: Chularat Wattanakit, Bangkok (TH); Wannaruedee Wannapakdee, Bangkok (TH); Duangkamon Suttipat, Bangkok (TH); Sitthiphong Pengpanich, Bangkok (TH)

(21) Appl. No.: 16/045,567

(22) Filed: Jul. 25, 2018

(30) Foreign Application Priority Data
Jul. 26, 2017 (TH) 1703001363

(51) Int. Cl. B01J 29/87; C01B 39/40; C01B 39/06; B01J 29/40; B01J 37/04; B01J 37/10; B01J 37/00; B01J 37/30; B01J 37/02; C07C 5/41

(52) U.S. Cl. CPC B01J 29/87; C01B 39/40; C01B 39/065; B01J 29/40; B01J 37/04; B01J 37/10; C07C 2529/40; B01J 37/30; B01J 37/0207; C07C 5/412; B01J 2229/183; B01J 2229/186; B01J 37/0018

(57) ABSTRACT

A process for preparing a hierarchical zeolite catalyst for aromatization of C5-C9 alkane that provides high conversion percentage of precursor to yields and high aromatics selectivity, wherein said process comprises the following steps:
(a) preparing a solution containing alumina compound, silica compound, and soft template;
(b) subjecting the mixture obtained from step (a) to hydrothermal process at determined time and temperature to form said mixture into the hierarchical zeolite;
(c) contacting the hierarchical zeolite obtained from step (b) with ammonium salt solution; and
(d) contacting the hierarchical zeolite obtained from step (c) with gallium salt solution;
wherein the soft template in step (a) is a quaternary phosphonium salt in which the mole ratio of the silica compound to the alumina compound in step (a) is in a range of 20 to 120 and the gallium salt in step (d) has gallium to zeolite ratio in a range of 0.5 to 5% by weight.

### TECHNICAL FIELD

Chemistry relates to the process for preparing a hierarchical zeolite catalyst for aromatization of C5-C9 alkane.

### BACKGROUND ART

Zeolite compound is the crystalline aluminosilicates compound that can be applied to several applications such as absorbent, ion exchanger, and heterogeneous catalysts because of the unique characters of zeolite such as acidity, heat and chemical stability, and shape selectivity. Therefore, zeolite is the very useful catalyst in petrochemical industry. However, there are several limitations in the application of the conventional zeolite catalyst in petrochemical processes such as low catalytic activity, fast deactivation, and complexity in regeneration steps of said catalyst. The main reason of these limitations of conventional zeolite is mass transfer and distribution because of its very small porous in zeolite structure (angstrom unit) in the large zeolite crystallite structure, resulting in critical mass transfer condition and difficulty of precursor to reach the reaction catalytic site. As a consequence, the accumulation of intermediates results in a high coke formation and high possibility of catalyst deactivation. From the above-mentioned factors, there have been attempts to design and develop the catalyst by improvement of zeolite structure to be suitable for reaction, especially in solving the limitations of mass transfer and distribution.

U.S. Pat. No. 7,824,657, US20130059722, and U.S. Pat. No. 8,951,498 disclose the synthesis of the hierarchical zeolite using several templates such as hard template and soft template for determining the hierarchical zeolite structure. However, said patent applications do not disclose the application of said zeolites as the catalyst. Moreover, there were limitations in structural improvement of the obtained catalyst having more than 1 catalytic site, including acidic catalytic active site and metallic catalytic active site, which are important catalyst's characteristics in many processes such as the conversion of hydrocarbon compounds into aromatic compounds via aromatization.

U.S. Pat. No. 4,861,933 and U.S. Pat. No. 4,304,686 disclose the process for preparing aromatics from aliphatic hydrocarbon using the conventional zeolite catalyst modified with gallium. However, it has been found that the selectivity of benzene, toluene, and xylene mixture (BTX) was low. Therefore, in the industrial scale, the addition processes are required for separation and purification to obtain the purified products.

US20130172648 discloses the catalyst and the process for preparing catalyst for the production of aromatics from propane. Said catalyst was prepared by treating the conventional zeolite with about 0.2 to 2% by weight of gallium and about 0.01 to 2% by weight of palladium or platinum metals, wherein said treatment was performed by impregnation and ion exchange methods. Nevertheless, the efficiency of said catalyst was not disclosed in the selectivity of para-xylene.

Wannapakdee et al., (RSC Advances, 2016, 6, 2875-2881) and Ogunronbi et al., (Journal of Molecular Catalysis A: Chemical, 2015, 406, 1-18) disclose the preparation of the hierarchical zeolite treated with gallium as the catalyst in the process for preparing aromatics from propane. However, there was no report on the use for aromatization of C5 or more atoms alkane such as pentane. Normally, the limitation of technology development of the conversion of C5 or more atoms alkane to aromatic compounds always have problem from the fast deactivation of the catalyst and the accumulation of coke formation within zeolite pores.

From all above-mentioned reasons, this invention aims to prepare the hierarchical zeolite for the improvement of zeolite structure to be suitable for the application of aromatization of C5-C9 alkane in order to provide high conversion and high aromatic selectivity.

### SUMMARY OF INVENTION

The present invention related to a process for preparing a hierarchical zeolite catalyst for aromatization of C5-C9 alkane providing high conversion and high aromatics selectivity, wherein said process comprises the following steps:

(a) preparing a solution containing alumina compound, silica compound, and soft template;

(b) subjecting the mixture obtained from step (a) to hydrothermal process at determined time and temperature to convert said mixture into the hierarchical zeolite;

(c) contacting the hierarchical zeolite obtained from step (b) with ammonium salt solution; and

(d) contacting the hierarchical zeolite obtained from step (c) with gallium salt solution;

wherein the soft template in step (a) is a quaternary phosphonium salt in which the mole ratio of the silica compound to the alumina compound in step (a) is in a range of 20 to 120 and the gallium salt in step (d) has gallium to zeolite ratio in a range of 0.5 to 5% by weight.

### BRIEF DESCRIPTION OF THE DRAWINGS

FIG. 1 shows the conversion of zeolite samples according to the invention with the addition of gallium metal by various methods.

FIG. 2 shows the aromatic selectivity of zeolite samples according to the invention with the addition of gallium by various methods.

FIG. 3 shows the conversion of zeolite samples according to the invention and the comparative samples.

FIG. 4 shows the aromatic selectivity of zeolite samples according to the invention and the comparative samples.

### DESCRIPTION OF THE INVENTION

The present invention relates to the process for preparing a hierarchical zeolite nanosheet catalyst for aromatization of C5-C9 alkane as described in the following embodiments.

Any aspect showed herein is meant to include its application to other aspects of this invention unless stated otherwise.

Technical terms or scientific terms used herein have definitions as by an ordinary person skilled in the art unless stated otherwise.

Any tools, equipment, methods, or chemicals named herein mean tools, equipment, methods, or chemicals being used commonly by an ordinary person skilled in the art unless stated otherwise that they are tools, equipment, methods, or chemicals specific only in this invention.

Use of singular noun or singular pronoun with “comprising” in claims or specification means “one” and including “one or more”, “at least one”, and “one or more than one”.

All compositions and/or methods disclosed and claims in this application aim to cover embodiments from any action, performance, modification, or adjustment without any experiment that significantly different from this invention, and obtain with object with utility and resulted as same as the present embodiment according to an ordinary person skilled in the art although without specifically stated in claims. Therefore, substitutable or similar object to the present embodiment, including any little modification or adjustment that clearly seen by an ordinary person skilled in the art should be construed as remains in spirit, scope, and concept of invention as appeared in appended claims.

Throughout this application, term “about” means any number that appeared or showed here that could be varied or deviated from any error of equipment, method, or personal using said equipment or method.

Hereafter, invention embodiments are shown without any purpose to limit any scope of the invention.

The present invention relates to the process for preparing a hierarchical zeolite nanosheet catalyst for aromatization of C5-C9 alkane, wherein said process comprises the following steps:

(a) preparing a solution containing alumina compound, silica compound, and soft template;

(b) subjecting the mixture obtained from step (a) to hydrothermal process at determined time and temperature to convert said mixture into the hierarchical zeolite;

(c) contacting the hierarchical zeolite obtained from step (b) with ammonium salt solution; and

(d) contacting the hierarchical zeolite obtained from step (c) with gallium salt solution;

characterized in that the soft template in step (a) is a quaternary phosphonium salt in which the mole ratio of the silica compound to the alumina compound in step (a) is in a range of 20 to 120 and the gallium salt in step (d) has gallium to zeolite ratio in a range of 0.5 to 5% by weight.

Preferably, the quaternary phosphonium salt is tetraalkylphosphonium selected from tetrabutylphosphonium hydroxide and tributyl hexadecyl phosphonium bromide, most preferably tetrabutylphosphonium hydroxide.

In one embodiment, the mole ratio of the silica compound to the alumina compound in step (a) is in the range of about 20 to 60.

In one embodiment, the gallium salt in step (d) has gallium to zeolite ratio in the range of about 0.5 to 1% by weight.

In one embodiment, the gallium salt is selected from gallium nitrate, gallium chloride, gallium bromide, gallium hydroxide, and gallium acetate, preferably is gallium nitrate.

In one embodiment, step (d) is performed by ion-exchange, impregnation, or chemical vapor deposition method. Preferably, it is performed by ion-exchange or impregnation method. Most preferably, it is performed by ion exchange method.

In one embodiment, the alumina compound in step (a) is aluminium isopropoxide or sodium aluminate.

In one embodiment, the silica compound in step (a) may be selected from tetraethyl orthosilicate or sodium silicate, preferably tetraethyl orthosilicate.

In one embodiment, step (b) is operated at the temperature in a range of about 120 to 160° C. for about 2 to 4 days.

In one embodiment, the said process for preparing the catalyst may further comprises drying and calcination steps.

Drying may be performed by general drying method using oven, vacuum drying, or stirred evaporation.

Calcination may be performed under atmospheric condition for about 1 to 10 hours and the temperature in the range of about 400 to 800° C., preferably about 4 to 6 hours at temperature of about 500 to 600° C.

In one embodiment, the catalyst obtained from the process for preparing according to the invention has hierarchical porous comprising micropore in a range of 0.4 to 0.6 nm, mesopore in the range of 2 to 10 nm, and macropore more than 50 nm, wherein the mesopore and macropore are 50% or more of the total pores.

In another embodiment, the present invention related to the use of catalyst obtained from the process for preparing according to the invention for aromatization of C5-C9 alkane to produce aromatics, preferably is for aromatization of pentane to produce aromatics, especially benzene, toluene, and xylene (BTX).

In one aspect, the aromatization may be performed by contacting fed alkane having 5 to 9 carbon atoms with catalyst prepared from the process according to the invention at the suitable conditions for the reaction. This can be operated in fixed bed system, moving bed system, fluidized bed system, or batch system.

The aromatization may be performed at the temperature in a range of about 400 to 800° C., preferably in the range of about 500 to 600° C. at the pressure under the atmospheric pressure to about 3,000 KPa, preferably in the range of about 100 to 500 KPa, most preferably at the atmospheric pressure.

The weight hourly space velocity (WHSV) of alkane feeding line in aromatization is in the range of about 1 to 30 hours⁻¹, preferably in the range of about 3 to 10 hour⁻¹.

Generally, any person skilled in this art can adjust the aromatization conditions to be suitable for type and composition of feed line, catalyst, and reactor system.

The following examples are demonstrated as one aspect of the invention, not for limiting the scope of this invention in any way.

Catalyst Preparation

The preparation of the catalyst according to the invention may be prepared by the following method.

The solution comprising aluminium isoproproxide and tetraethylorthosilicate was prepared, wherein the mole ratio of silica to alumina was 60 and 120 using tetrabutylphosphonium hydroxide as template of zeolite. The solution was heated at the temperature about 120-160° C. for about 2-4 days. Then, the prepared solution was washed with deionized water until the pH of the washing water was lower than 9 and the obtained substance was dried at the temperature about 100-120° C. for 20-24 hours. Then, calcination was performed in order to remove the template at the temperature about 500-650° C. for about 8-12 hours. The hierarchical zeolite was obtained as white powder.

Said zeolite was ion exchanged with ammonium chloride solution by subjecting about 1 g of obtained hierarchical zeolite to ion exchanging with about 100 mL of about 0.1 mole/L of ammonium chloride solution at the temperature about 80° C. for about 2 hours. Then, the obtained solution was filtered and washed until the pH was neutral. Then, the obtained substance was calcinated at the temperature about 550° C. for about 6 hours.

The zeolite obtained from contacting with the above ammonium chloride solution was contacted with gallium nitrate solution by ion exchange or impregnation method. The details of each method were as following.

Contacting with Gallium Nitrate Solution by Ion Exchange Method

The ion exchange operation could be performed by contacting about 1 g of zeolite obtained from above process to about 20 mL of gallium nitrate solution at the gallium to zeolite ratio about 1% by weight. Then, the obtained mixture was stirred at the temperature about 80° C. for about 2 hours. The obtained mixture was washed with distilled water and dried at the temperature about 100° C. for about 12 hours. Then, the obtained substance was calcinated at the temperature about 550° C. for about 6 hours.

Contacting with Gallium Nitrate Solution by Impregnation Method

The impregnation method could be performed by adding about 20 mL of gallium nitrate solution into about 1 g of zeolite obtained from the above process. The gallium to zeolite ratio was about 1% by weight. Then, the obtained mixture was stirred for about 30 minutes to 12 hours. Then, the obtained substance was dried with rotary evaporator and calcined at the temperature about 550° C. for about 6 hours.

Adding of Gallium Metal by In-Situ Synthesis Method

The addition of gallium metal by in-situ synthesis method could be performed by further adding of gallium nitrate solution at the ratio of 1% by weight into mixture comprising alumina compound, silica compound, and zeolite template in the step for preparing the hierarchical zeolite as described above.

Comparative Sample Cat A (Ga(exc)ZSM5-Con-120)

The ZSM-5 zeolite with mole ratio of silica to alumina about 120 that had been synthesized according to the method disclosed by Hensen et al., (Catalysis Today, 2011, 168, 96-111) was brought to contact with ammonium chloride solution and gallium nitrate solution by ion exchanging method described above.

Comparative Sample Cat B (Ga(exc)ZSMS-Con-60)

The ZSM-5 zeolite with mole ratio of silica to alumina about 60 that had been synthesized according to the method disclosed by Hensen et al., (Catalysis Today, 2011, 168, 96-111) was brought to contact with ammonium chloride solution and gallium nitrate solution by ion exchanging method described above.

Sample According to the Invention Cat 1 (Ga(exc)ZSM5-NS-120)

The sample according to the invention Cat 1 was prepared by the method according to the invention as described above using mole ratio of silica to alumina of 120 and used the contacting of gallium nitrate with ion exchanging method described above.

Sample According to the Invention Cat 2 (Ga(exc)ZSM5-NS-60)

The sample according to the invention Cat 2 was prepared by the method according to the invention as described above using mole ratio of silica to alumina of 60 and used the contacting of gallium nitrate by ion exchanging method described above.

Sample According to the Invention Cat 3 (Ga(impreg)ZSM5-NS-120)

The sample according to the invention Cat 3 was prepared by the method according to the invention as described above using mole ratio of silica to alumina of 120 and used the contacting of gallium nitrate with impregnation method described above.

Sample According to the Invention Cat 4 (Ga(impreg)ZSM5-NS-60)

The sample according to the invention Cat 4 was prepared by the method according to the invention as described above using mole ratio of silica to alumina of 60 and used the contacting of gallium nitrate with impregnation method described above.

Aromatization Test

The aromatization test may be performed by the following conditions.

The aromatization was operated in the fixed-bed reaction with about 0.2 g of catalyst. Prior to the reaction, the catalyst was contacted with hydrogen under about 2-10% by volume of nitrogen with flow rate about 2 mL/min for about 1 hour. Then, pentane was fed at the flow rate of about 1-3 g/hr. The reaction was operated continuously at the temperature about 500-550° C. at the atmospheric pressure and the weight hourly space velocity (WHSV) about 5 hour⁻¹.

Then, the reaction was followed by measuring the conversion and product compositions at reaction time by gas chromatography connected to the outlet of the fixed bed reactor using flame ionization detector (FID) as the detector and the HP-AL/S and GASPRO capillary column for the analysis of each said composition.

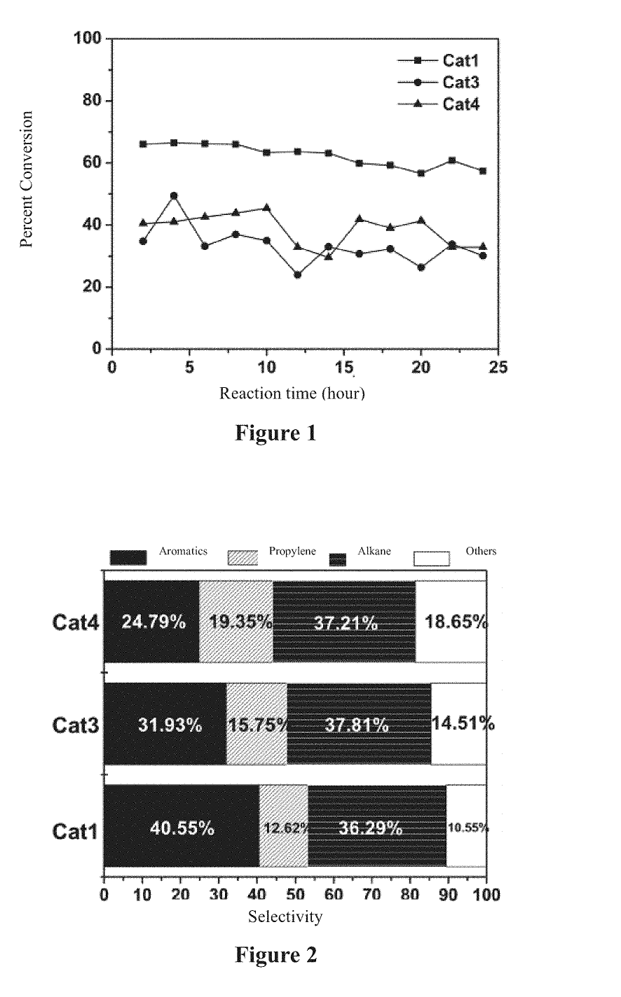

Table 1 shows the physical properties of the hierarchical zeolite prepared from the invention with the mole ratio of silica to alumina about 60 and 120, wherein said zeolite was not contacted with ammonium salt solution and gallium salt solution. From the table, it was found that the zeolite prepared from the invention comprises micropore, mesopore, and macropore, wherein the mesopore and macropore was more than 80% of total pores. This was 20 times higher than conventional zeolite. This result represents the hierarchical porous. Moreover, to show crystalline structure, the obtained substance was tested by transmission electron microscopy (TEM). Results were shown in FIG. 1 that the zeolite prepared from the invention was the thin nanosheet having particle size in a range of about 120-200 nm.

TABLE 1
The specific area and porous properties of each zeolite

| | S_{BET} | S_{Ext} | S_{Ext}/ | Vₜₒₜ | V_{micro} | V_{meso+macro} |
|---|---|---|---|---|---|---|
| | (m²/ | (m²/ | S_{BET} | (cm³/ | (cm³/ | (cm³/ |
| Sample | g) | g) | (%) | g) | g) | g) |
| Hierarchical zeolite | 478 | 161 | 34 | 1.02 | 0.12 | 0.90 |
| at silica to alumina | | | | | | |
| mole ratio of 60 | | | | | | |
| Hierarchical zeolite | 558 | 295 | 53 | 1.10 | 0.11 | 0.99 |
| at silica to alumina | | | | | | |
| mole ratio of 120 | | | | | | |
| Conventional zeolite | 408 | 23 | 5.6 | 0.22 | 0.18 | 0.04 |
| at silica to alumina | | | | | | |
| mole ratio of 60 | | | | | | |

Note:
S_{BET}: BET specific surface area;
Sₑₓₜ: external surface area;
Vₜₒₜₐₗ: total pore volume;
V_{micro}: micropore volume;
V_{meso+macro}: mesopore and macropore volume

Effect of the Hierarchical Zeolite on the Metal Addition Efficacy

To study the effect of the hierarchical zeolite on the metal addition efficiency, the conventional zeolite with silica to alumina ratio of 120 was compared with the zeolite prepared by the process according to the invention using ion exchanging method of contacting zeolite to the gallium salt solution. The results were shown in table 2.

Table 2 shows the % by weight of gallium measured with x-ray fluorescence (XRF) of zeolite sample prepared from method according to the invention and the conventional zeolite under the same testing conditions. It was found that the hierarchical structure results in gallium metal to be exchanged in zeolite structure better than the conventional zeolite without the hierarchical structure.

TABLE 2
The % by weight of gallium of each zeolite

| Sample | Measured gallium |
|---|---|
| Sample according to the invention Cat 1 | 1.0% by weight |
| Comparative sample Cat A | 293 ppm |

Effect of the Addition of Gallium Metal in the Hierarchical Zeolite

To study the effect of the addition of gallium in the hierarchical zeolite on the efficiency of said zeolite as the aromatization catalyst, the various addition methods of gallium such as ion exchange, impregnation, and in-situ synthesis were studied. Results were shown in FIG. 1 and FIG. 2. It was found that the sample according to the invention Cat 1 prepared from process according to the invention by ion exchange gave highest efficiency for the aromatization in both reactivity and selectivity of aromatics.

Effect of the Hierarchical Porous Structure on the Aromatization Efficiency

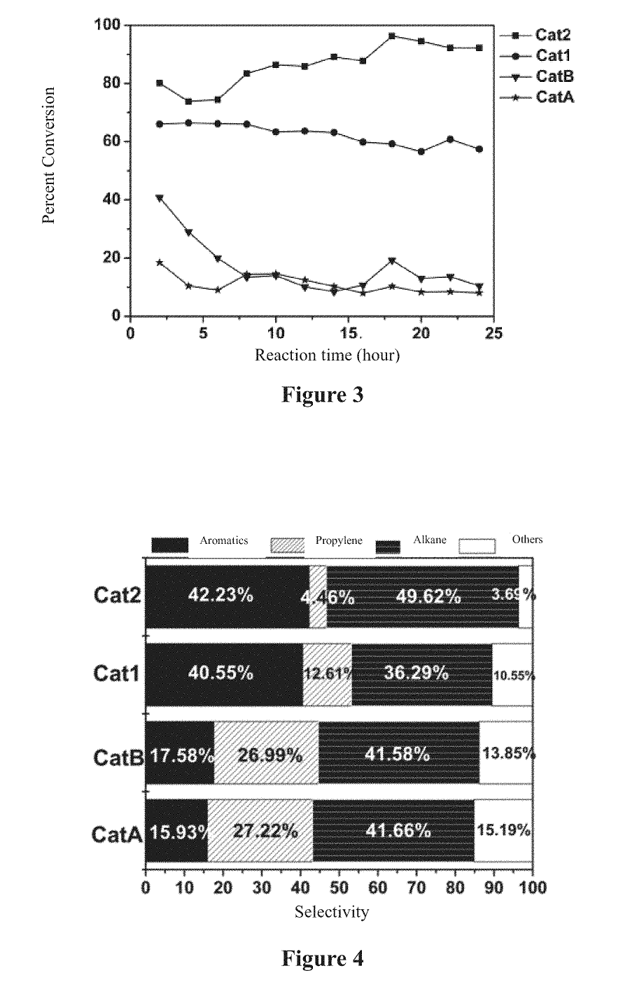

To study of the effect of the hierarchical porous structure on the efficiency of said zeolite as the aromatization catalyst, the zeolites according to the invention were compared with the comparative sample using the conventional zeolite. The results were shown in FIG. 3 and FIG. 4.

From FIG. 3 and FIG. 4, it was found that the sample according to the invention Cat 1 and Cat 2 prepared from the process according to the invention showed better pentane conversion and higher aromatics selectivity than the conventional zeolite.

From the results above, it can be said that the catalyst prepared from the process according to the invention gave high conversion and high aromatics selectivity for the aromatization of C5 to C9 alkane as indicated in the objectives of this invention.

### Preferred Embodiment of the Invention

Preferred embodiment of the invention is as provided in the description of the invention.

1. A process for preparing a hierarchical zeolite catalyst for aromatization of C5-C9 alkane, wherein said process comprises the following steps:
(a) preparing a solution containing alumina compound, silica compound, and soft template;
(b) subjecting the mixture obtained from step (a) to hydrothermal process at determined time and temperature to form said mixture into the hierarchical zeolite;
(c) contacting the hierarchical zeolite obtained from step (b) with ammonium salt solution; and
(d) contacting the hierarchical zeolite obtained from step (c) with gallium salt solution;
characterized in that the soft template in step (a) is a quaternary phosphonium salt in which the mole ratio of the silica compound to the alumina compound in step (a) is in a range of 20 to 120 and the gallium salt in step (d) has gallium to zeolite ratio in a range of 0.5 to 5% by weight.

2. The process for preparing according to claim 1, wherein the quaternary phosphonium salt is tetraalkylphosphonium selected from tetrabutylphosphonium hydroxide and tributyl hexadecyl phosphonium bromide.

3. The process for preparing according to claim 2, wherein the quaternary phosphonium salt is tetrabutylphosphonium hydroxide.

4. The process for preparing according to claim 1, wherein the mole ratio of the silica compound to the alumina compound in step (a) is in the range of 20 to 60.

5. The process for preparing according to claim 1, wherein the gallium salt in step (d) has gallium to zeolite ratio in the range of 0.5 to 1% by weight.

6. The process for preparing according to claim 1, wherein the gallium salt is selected from gallium nitrate, gallium chloride, gallium bromide, gallium hydroxide, and gallium acetate.

7. The process for preparing according to claim 1, wherein step (d) is performed by ion-exchange or impregnation method.

8. The process for preparing according to claim 1, wherein the alumina compound in step (a) is aluminium isopropoxide or sodium aluminate.

9. The process for preparing according to claim 1, wherein the silica compound in step (a) is tetraethyl orthosilicate.

10. A catalyst obtained from the process for preparing according to claim 1, wherein said catalyst is the hierarchical catalyst comprising micropore in a range of 0.4 to 0.6 nm, mesopore in a range of 2 to 10 nm, and macropore more than 50 nm, in which the mesopore and macropore are 50% or more of the total pores.

11. The process for preparing according to claim 5, wherein the gallium salt is selected from gallium nitrate, gallium chloride, gallium bromide, gallium hydroxide, and gallium acetate.

12. A catalyst obtained from the process for preparing according to claim 2, wherein said catalyst is the hierarchical catalyst comprising micropore in a range of 0.4 to 0.6 nm, mesopore in a range of 2 to 10 nm, and macropore more than 50 nm, in which the mesopore and macropore are 50% or more of the total pores.

13. A catalyst obtained from the process for preparing according to claim 3, wherein said catalyst is the hierarchical catalyst comprising micropore in a range of 0.4 to 0.6 nm, mesopore in a range of 2 to 10 nm, and macropore more than 50 nm, in which the mesopore and macropore are 50% or more of the total pores.

14. A catalyst obtained from the process for preparing according to claim 4, wherein said catalyst is the hierarchical catalyst comprising micropore in a range of 0.4 to 0.6 nm, mesopore in a range of 2 to 10 nm, and macropore more than 50 nm, in which the mesopore and macropore are 50% or more of the total pores.

15. A catalyst obtained from the process for preparing according to claim 5, wherein said catalyst is the hierarchical catalyst comprising micropore in a range of 0.4 to 0.6 nm, mesopore in a range of 2 to 10 nm, and macropore more than 50 nm, in which the mesopore and macropore are 50% or more of the total pores.

16. A catalyst obtained from the process for preparing according to claim 6, wherein said catalyst is the hierarchical catalyst comprising micropore in a range of 0.4 to 0.6 nm, mesopore in a range of 2 to 10 nm, and macropore more than 50 nm, in which the mesopore and macropore are 50% or more of the total pores.

17. A catalyst obtained from the process for preparing according to claim 7, wherein said catalyst is the hierarchical catalyst comprising micropore in a range of 0.4 to 0.6 nm, mesopore in a range of 2 to 10 nm, and macropore more than 50 nm, in which the mesopore and macropore are 50% or more of the total pores.

18. A catalyst obtained from the process for preparing according to claim 8, wherein said catalyst is the hierarchical catalyst comprising micropore in a range of 0.4 to 0.6 nm, mesopore in a range of 2 to 10 nm, and macropore more than 50 nm, in which the mesopore and macropore are 50% or more of the total pores.

19. A catalyst obtained from the process for preparing according to claim 9, wherein said catalyst is the hierarchical catalyst comprising micropore in a range of 0.4 to 0.6 nm, mesopore in a range of 2 to 10 nm, and macropore more than 50 nm, in which the mesopore and macropore are 50% or more of the total pores.

20. A catalyst obtained from the process for preparing according to claim 11, wherein said catalyst is the hierarchical catalyst comprising micropore in a range of 0.4 to 0.6 nm, mesopore in a range of 2 to 10 nm, and macropore more than 50 nm, in which the mesopore and macropore are 50% or more of the total pores.
